(19) United States
(12) Patent Application Publication
(10) Pub. No.: US 2004/0180908 A1
(43) Pub. Date: Sep. 16, 2004

(54) Adenosine derivative in polymorph ii form

(76) Inventor: Paula King, Ware (GB)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/481,612

(22) PCT Filed: Jun. 19, 2002
(86) PCT No.: PCT/GB02/02841

(30) Foreign Application Priority Data
Jun. 20, 2001 (GB) 0115178.6

(51) Int. Cl. A61K 31/52; C07D 473/14

(52) U.S. Cl. 514/263.2; 544/277

(57) ABSTRACT

(2S,3S,4R,5R)-2-(5-tert-butyl-[1,3,4]-oxadiazol-2-yl)-5-[6-(4-chloro-2-fluorophenylamino)-9H-purin-9-yl]-tetrahydrofuran-3,4-diol in polymorphic form.

The present invention relates to heterocyclyl substituted adenosine derivatives. More particularly the invention is concerned with a particular physical form of (2S,3S,4R,5R)-2-5-tert-butyl-[1,3,4]-oxadiazol-2-yl)-5-[6-4-chloro-2-fluorophenylamino)-9H-purin-9-yl]-tetrahydrofuran-3,4-diol, pharmaceutical formulations thereof and its use in therapy.

WO99/67262 (Glaxo Group Limited) discloses certain heterocyclyl adenosine derivatives including (2S,3S,4R,5R)-2-(5-tert-butyl-[1,3,4]-oxadiazol-2-yl)-5-[6-(4-chloro-2-fluorophenylamino)-9H-purin-9-yl]-tetrahydrofuran-3,4-diol, Example 14 of WO99/67262, the structure of which is indicated below as the compound of formula (A):

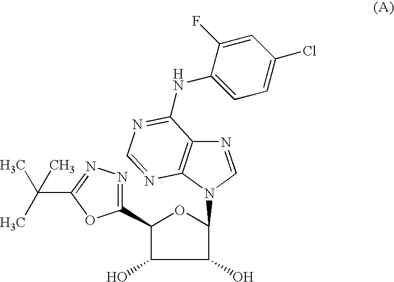

The preparation of the compound of formula (A) is described in WO99/67262. The compound of formula (A) may be prepared by the reaction of 4-chloro-2-fluoroaniline with an appropriate purinyl derivative having a suitable leaving group in the 6-position of the purine ring, optionally in the presence of a solvent at elevated temperatures. Alternatively the compound of formula (A) may be prepared by treating 9-{(3aR,4R,6S,6aR)-6-[5-tert-butyl-1,3,4-oxadiazol-2-yl]-2,2-dimethyltetrahydrofuro[3,4-d]-[1,3]dioxol-4-yl}-N-(4-chloro-2-fluorophenyl)-9H-purin-6-amine with trifluoroacetic acid followed by treatment with sodium bicarbonate. Extraction of the product into ethyl acetate followed by evaporation in vacuo provides the compound of formula (A) as a buff solid.

We have now surprisingly found that the compound of formula (A) can be obtained in polymorphic form.

There is thus provided as a first aspect of the invention (2S,3S,4R,5R-2-(5-tert-butyl-[1,3,4]-oxadiazol-2-yl)-5-[6-(4-chloro-2-fluorophenylamino)-9H-purin-9-yl]-tetrahydrofuran-3,4-diol in polymorphic form.

We have further found that the compound of formula (A) may also be crystallised in the form of polymorphic form II (hereinafter Polymorph II).

There is thus provided in a yet further aspect of the invention (2S,3S,4R,5R)-2-(5-tert-butyl-[1,3,4]-oxadiazol-2-yl)-5-[6-(4-chloro-2-fluorophenylamino)-9H-purin-9-yl]-tetrahydrofuran-3,4-diol as Polymorph II.

Polymorph II exhibits particular stability at elevated temperatures, for example temperatures in excess of 70° C.

Polymorph II may be useful in the preparation of pharmaceutical formulations which may involve temperatures above ambient temperatures.

In a preferred aspect the invention provides (2S,3S,4R,5R-2-(5-tert-butyl-[1,3,4]-oxadiazol-2-yl)-5-[6-4-chloro-2-fluorophenylamino)-9H-purin-9-yl]-tetrahydrofuran-3,4-diol in the form of Polymorph II as herein defined substantially free of impurities.

In a further preferred aspect the invention (2S,3S,4R,5R)-2-5-tert-butyl-[1,3,4]-oxadiazol-2-yl)-5-[6-(4-chloro-2-fluorophenylamino)-9H-purin-9-yl]-tetrahydrofuran-3,4-diol in the form of Polymorph II as herein defined substantially free of alternative polymorphs.

By “substantially free” is meant containing less than 10%, preferably less than 5%, more preferably less than 2%, of alternative polymorph or impurity.

(2S,3S,4R,5R-2-(5-tert-Butyl-[1,3,4]-oxadiazol-2-yl)-5-[6-(4-chloro-2-fluorophenylamino)-9H-purin-9-yl]-tetrahydrofuran-3,4-diol may be prepared in polymorphic form by crystallisation of the compound under suitable conditions.

Polymorph II may be prepared substantially free from other polymorphs by controlling crystallisation conditions.

In general, (2S,3 S,4R,5R)-2-(5-tert-butyl-[1,3,4]-oxadiazol-2-yl)-5-[6-(4-chloro-2-fluorophenylamino)-9H-purin-9-yl]-tetrahydrofuran-3,4-diol in the form of Polymorph II may be obtained by crystallisation of the compound by heating in methyl isobutyl ketone at reflux (117-118° C.) and allowing to cool to ambient temperature, for example 15-25° C.

Polymorph II may also be prepared by dissolving (2S,3S,4R,5R)-2-5-tert-butyl-[1,3,4]-oxadiazol-2-yl)-5-[6-4-chloro-2-fluorophenylamino)-9H-purin-9-yl]-tetrahydrofuran-3,4-diol in methyl isobutyl ketone at reflux, filtering, concentrating the filtrate, cooling to 45-70° C., preferably 50-55° C. and collecting Polymorph H by filtration.

Alternatively Polymorph II is prepared by dissolving (2S,3S,4R,5R-2-(5-tert-Butyl-[1,3,4]oxadiazol-2-yl)-O-[6-(4-chloro-2-fluorophenylamino)-9H-purin-9-yl]-tetrahydrofuran-3,4-diol in N,N-dimethylformamide and water wherein the N,N-dimethylformamide:water ratio is from 2:1 to 1:2, optionally treating with decolourising charcoal, adjusting the temperature to greater than 35° C., and optionally seeding with polymorph II. Optionally, toluene may be added prior to collecting the resulting solid.

Interconversion of one polymorph to another can occur under certain circumstances.

The methods for the preparation of polymorphic material, and in particular methods for the preparation of Polymorph II, described herein constitute further aspects of the present invention.

Polymorph II has been characterised by X-ray powder diffraction (XRPD) studies and Raman spectroscopy.

Polymorph II is characterised by having peaks in its Raman spectra at 3424, 1615 and 92 cm⁻¹.

Raman peaks are quoted to the nearest cm−1.

Polymorph II is characterised by having an XRPD pattern with signals at 4.74, 5.34, 6.63, 7.87, 8.31, 8.93, 10.71, and 13.98 (degrees 2-theta).

The skilled person will recognise that XRPD peak positions are affected by differences in sample height. The peak positions quoted herein are thus subject to a variation of +/−0.15 degrees 2-theta.

This invention further provides for a pharmaceutical composition comprising (2S,3S,4R,5R)-2-(5-tert-butyl-[1,3,4]oxadiazol-2-yl)-5-[6-4-chloro-2-fluorophenylamino)-9H-purin-9-yl]-tetrahydrofuran-3,4-diol in polymorphic form, and a pharmaceutically acceptable carrier and/or excipient.

Suitable pharmaceutically acceptable carriers and excipients are described in WO 99/967262.

(2S,3 S,4R,5R-2-5-tert-Butyl-[1,3,4]-oxadiazol-2-yl)-5-[6-(4-chloro-2-fluorophenylamino)-9H-purin-9-yl]-tetrahydrofuran-3,4-diol in polymorphic form may be used for decreasing plasma free fatty acid concentration; reducing heart rate; or treating ischemic heart disease, peripheral vascular disease, stroke, pain, CNS disorder, or sleep apnoea, as described in WO 99/67262.

(2S,3 S,4R,5R-2-(5-tert-Butyl-[1,3,4]-oxadiazol-2-yl)-5-[6-(4-chloro-2-fluorophenylamino)-9H-purin-9-yl]-tetrahydrofuran-3,4-diol in polymorphic form may be used in the manufacture of a medicament for use in decreasing plasma free fatty acid concentration; reducing heart rate; or treating ischemic heart disease, peripheral vascular disease, stroke, pain, CNS disorder, or sleep apnoea, as described in WO 99/67262.

WO 99/67262 (Glaxo Group Limited) is incorporated by reference herein as though fully set forth.

The following examples illustrate the invention but are not intended as a limitation thereof.

### EXAMPLES

(2S,3S,4R,5R-2-(5-tert-Butyl-[1,3,4]oxadiazol-2-yl)-5-[6-(4-chloro-2-fluorophenylamino)-9H-purin-9-yl]-tetrahydrofuran-3,4-diol was prepared according to the methods described in WO99/67262.

### Example 1

### Preparation of Polymorph II

(2S,3S,4R,5R)-2-5-tert-Butyl-[1,3,4]oxadiazol-2-yl)-5-[6-(4-chloro-2-fluorophenylamino)-9H-purin-9-yl]-tetrahydrofuran-3,4-diol (10 g) was taken up in methyl isobutyl ketone (MIBK, 170 mL) and the mixture heated to reflux to effect dissolution. The solution was then cooled to ambient over ca 30 mins (crystallisation commenced at ca. 70° C.) and the thick slurry stirred fo a further hour. The matted crystals were then filtered off, washed with cold MIBK (1×15 mL) and dried in vacuo at 60° C. Yield: 83%.

### Example 2

### Preparation of Polymorph B

(2S,3S,4R,5R-2-(5-tert-Butyl-[1,3,4]oxadiazol-2-yl)-5-[6-(4-chloro-2-fluorophenylamino)-9H-purin-9-yl]-tetrahydrofuran-3,4-diol (165.8 g) was dissolved in MIBK (3800 mL) at reflux. The resulting solution was filtered and the filter washed with MIBK (415 mL). The combined filtrate and wash were re-heated to reflux and MIBK (1520 mL) was removed by distillation under reduced pressure. The residue was cooled to 50° C. and the product was collected by filtration, washed with MIBK and then dried in vacuo at 40° C. to give Polymorph II as an off white solid (130.9 g, 75% recovery).

X-Ray Powder Diffraction

The sample preparation and acquisition conditions were as follows:

Samples were lightly ground and packed into silicon cup with a 12 mm (diameter)×0.5 mm cavity. Data were acquired using a Bruker D8 Advance X-Ray diffractometer configured with a Cu anode, primary and secondary Soller slits, secondary monochromator and scintillation counter. The generator was operated at 40 kV 40 mA. Variable divergence and antiscatter slits were set at 12 mm irradiated area, and the detector slit was set at 0.1 mm. A locked coupled step scan with 0.02 degrees 2-theta step was used. The sample was rotated.

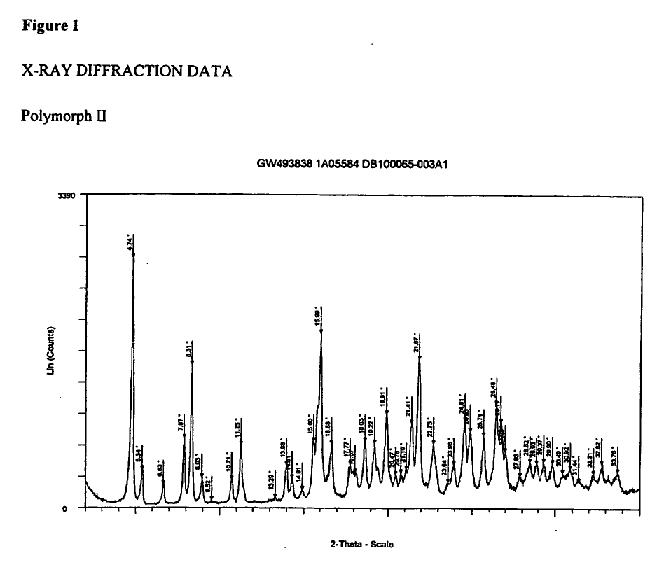

Data obtained for Polymorph II are shown in FIG. 1.

Raman Spectroscopy

Raman spectra were acquired using a Nicolet 960 ESP FT-Raman spectrometer. Samples were held in glass vials; spectra of 5 different points on a sample were averaged. Data collection parameters include: Laser power: 400 mW, Resolution: 4 cm⁻¹, Sample gain: 1.0, Detector: InGaAs, Beamsplitter: CaF2, Correction: none, Zero filling: none, Apodization: Happ-Genzel, Phase correction: Power spectrum.

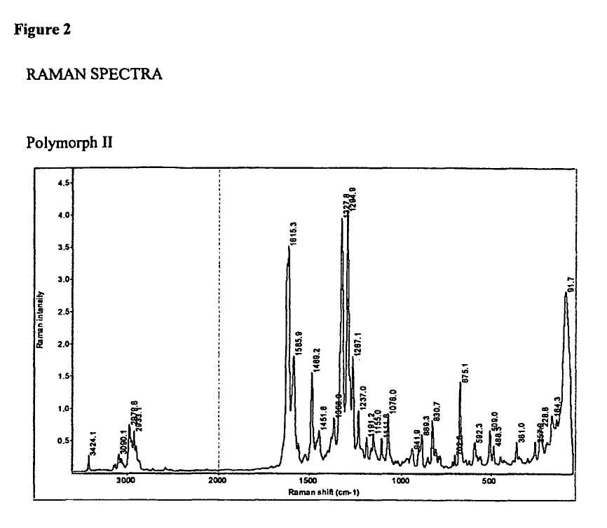

A Raman spectrum of Polymorphs II is shown in FIG. 2.

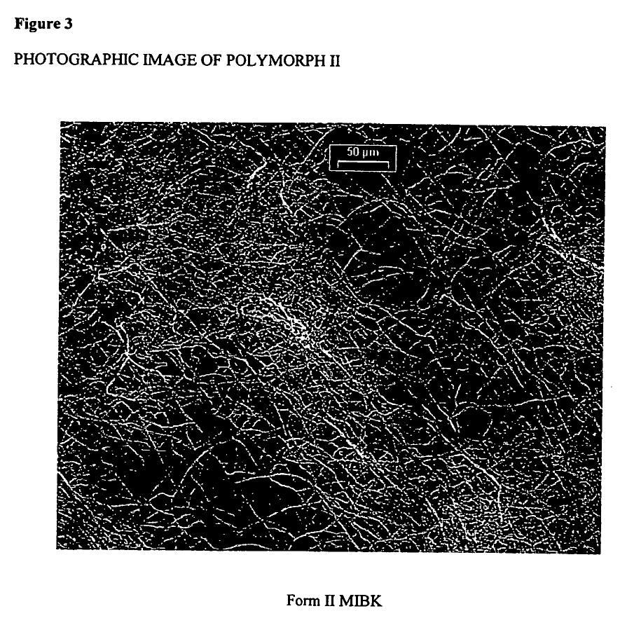

A photographic image of Polymorph II is shown in FIG. 3.

The application of which this description and these claims form a part may be used as a basis for priority in respect of any subsequent application. The claims of such subsequent application may be directed to any novel feature or combination of features relating to the invention described herein. They may take the form of product, process or use claims and may include, by way of example and without limitation, the claims that follow.

1. (2S,3S,4R,5R)-2-(5-tert-Butyl-[1,3,4]-oxadiazol-2-yl)-5-[6-(4-chloro-2-fluorophenylamino)-9H-purin-9-yl]-tetrahydrofuran-3,4-diol in polymorphic form.

2. A polymorphic form according to claim 1 wherein the polymorphic form is Polymorph II.

3. A pharmaceutical formulation comprising a polymorphic form according to claim 1 or claim 2, and a pharmaceutically acceptable carrier and/or excipient.

4. A polymorphic form according to to claim 1 or claim 2 for use in decreasing plasma free fatty acid concentration; reducing heart rate; or treating ischemic heart disease, peripheral vascular disease, stroke, pain, CNS disorder, or sleep apnoea.

5. Use of a polymorphic form according to to claim 1 or claim 2 in the manufacture of a medicament for use in decreasing plasma free fatty acid concentration; reducing heart rate; or
treating ischemic heart disease, peripheral vascular disease, stroke, pain, CNS disorder, or sleep apnoea.

6. (2S,3S,4R,5R)-2-(5-tert-butyl-[1,3,4]-oxadiazol-2-yl)-5-[6-(4-chloro-2-fluorophenylamino)-9H-purin-9-yl]-tetrahydrofuran-3,4-diol in polymorphic form substantially as described herein in the specification and/or examples.
